(19) United States
(12) Patent Application Publication
(10) Pub. No.: US 2026/0308081 A1
(43) Pub. Date: Oct. 8, 2026

(54) METHOD OF STARTING UP A FISCHER-TROPSCH PLANT

(71) Applicant: JOHNSON MATTHEY DAVY TECHNOLOGIES LIMITED, London (GB)

(72) Inventors: Chi Ying CHAN, London (GB); Amelia Lorna Solveig COOK, London (GB)

(21) Appl. No.: 19/479,620

(22) PCT Filed: Apr. 12, 2024
(86) PCT No.: PCT/GB2024/050966

§ 371 Date: Oct. 28, 2025

(30) Foreign Application Priority Data
Jun. 15, 2023 (GB) 2308962.6

(51) Int. Cl. C07C 1/04

(52) U.S. Cl. CPC C07C 1/047; C07C 1/042

(57) ABSTRACT

A method of starting up a Fischer-Tropsch plant, the method comprising: providing a Fischer-Tropsch plant comprising a fixed-bed Fischer-Tropsch unit downstream of a reverse-water-gas-shift unit; providing a feed comprising hydrogen and carbon dioxide; passing the feed to the reverse-water-gas-shift unit to produce a syngas enriched in carbon monoxide; providing a first water source; generating a first steam from the first water source using heat from the syngas to thereby form a cooled syngas; passing at least some of the first steam to the Fischer-Tropsch unit to increase the temperature of the Fischer-Tropsch unit to a first operating temperature of the Fischer-Tropsch unit.

### FIELD OF THE INVENTION

The invention relates to a method of starting up a Fischer-Tropsch plant, and a method of starting-up a Fischer-Tropsch plant and then operating the Fischer-Tropsch plant to produce a hydrocarbon product.

### BACKGROUND OF THE INVENTION

Examples of Fischer-Tropsch plants and their operation are described in WO2021140227A1, WO2018146276A1, WO2017037175A1, WO2015140100A1, WO2015140099A1, WO2015010939A1 and WO2009128865A1.

The Fischer-Tropsch process is a collection of chemical reactions that converts a mixture of carbon monoxide and hydrogen (also known as “syngas”) into liquid hydrocarbons. These reactions occur in the presence of metal catalysts, typically at temperatures of 150-300° C. and pressures of one to several tens of atmospheres. The Fischer-Tropsch process involves a series of chemical reactions that produce a variety of hydrocarbons, ideally having the formula (CₙH₂ₙ₊₂). The more useful reactions produce alkanes as follows:

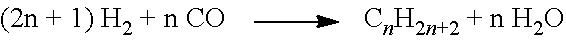

where n is typically 1-100 or higher. The formation of methane (n=1) is unwanted. Most of the alkanes produced tend to be straight-chain and are suitable to be upgraded to produce middle distillate fuels such as diesel and jet fuel. In addition to alkane formation, competing reactions give small amounts of alkenes, as well as alcohols and other oxygenated hydrocarbons. The Fischer-Tropsch reaction is a highly exothermic reaction due to a standard reaction enthalpy (ΔH) of −165 kJ/mol CO combined.

In a typical Fischer-Tropsch plant, the syngas fed to the Fischer-Tropsch unit is typically prepared by subjecting a feed gas comprising hydrogen and carbon dioxide to a reverse-water-gas-shift reaction to convert some of the carbon dioxide and hydrogen to carbon monoxide and water.

The start-up of a typical Fischer-Tropsch plant involves increasing the temperature of the Fischer-Tropsch unit to an operating temperature suitable for carrying out the Fischer-Tropsch reaction at a favourable reaction rate, conversion rate and/or selectivity. This typically involves passing steam to the Fischer-Tropsch unit. This increases the complexity of the plant in view of the need to have an external source of steam, and also increases the cost.

The present invention seeks to tackle at least some of the problems associated with the prior art or at least to provide a commercially acceptable alternative solution thereto.

### SUMMARY OF THE INVENTION

One aspect of the present disclosure is directed to a method of starting-up a Fischer-Tropsch plant, the method comprising:
providing a Fischer-Tropsch plant comprising a fixed-bed Fischer-Tropsch unit downstream of a reverse-water-gas-shift unit;
providing a feed comprising hydrogen and carbon dioxide;
passing the feed to the reverse-water-gas-shift unit to produce a syngas enriched in carbon monoxide;
providing a first water source;
generating a first steam from the first water source using heat from the syngas to thereby form a cooled syngas;
passing at least some of the first steam to the Fischer-Tropsch unit to increase the temperature of the Fischer-Tropsch unit to a first operating temperature of the Fischer-Tropsch unit.

Another aspect of the present disclosure is directed to a method of starting up a Fischer-Tropsch plant and then operating the Fischer-Tropsch plant to produce a hydrocarbon product, the method comprising:
starting up a Fischer-Tropsch plant using the method as defined above; and
once the Fischer-Tropsch plant has reached the first operating temperature, operating the Fischer-Tropsch plant by passing the cooled syngas to the Fischer-Tropsch unit to produce a hydrocarbon product.

### BRIEF DESCRIPTION OF THE DRAWINGS

FIG. 1 shows a flow diagram of an example of a method according to the present invention.

### DETAILED DESCRIPTION OF THE INVENTION

In a first aspect, the present disclosure is directed to a method of starting up a Fischer-Tropsch plant, the method comprising:
providing a Fischer-Tropsch plant comprising a fixed-bed Fischer-Tropsch unit downstream of a reverse-water-gas-shift unit;
providing a feed comprising hydrogen and carbon dioxide;
passing the feed to the reverse-water-gas-shift unit to produce a syngas enriched in carbon monoxide;
providing a first water source;
generating a first steam from the first water source using heat from the syngas to thereby form a cooled syngas;
passing at least some of the first steam to the Fischer-Tropsch unit to increase the temperature of the Fischer-Tropsch unit to a first operating temperature of the Fischer-Tropsch unit.

Each aspect or embodiment as defined herein may be combined with any other aspect(s) or embodiment(s) unless clearly indicated to the contrary. In particular, any features indicated as being preferred or advantageous may be combined with any other feature indicated as being preferred or advantageous.

Advantageously, the method of the present invention may enable the Fischer-Tropsch unit to reach an operating temperature without the use of an external source of steam. This reduces the complexity of the method and avoids the need to employ additional equipment to generate the steam. Using heat from the syngas to generate the steam reduces the energy requirement of the method, thereby reducing the cost and environmental impact.

The method is a method of starting up a Fischer-Tropsch plant. The wording “starting up a Fischer-Tropsch plant” as used herein may encompass a method of increasing the Fischer-Tropsch unit to a temperature sufficient to undergo a Fischer-Tropsch reaction.

The method comprises providing a Fischer-Tropsch plant comprising a fixed-bed Fischer-Tropsch unit. Fixed-bed Fischer-Tropsch units are known in the art.

The fixed-bed Fischer-Tropsch unit is provided downstream of a reverse-water-gas-shift unit. Reverse-water-gas-shift units are known in the art. Under the reverse-water-gas-shift reaction, carbon dioxide and hydrogen are converted as follows:

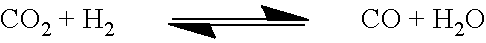

In the method, a portion of the carbon dioxide and a portion of the hydrogen are converted to carbon monoxide and water. As will be appreciated, not all of the hydrogen is converted. This is because of the need for the syngas to contain hydrogen so as to undergo, for example, a subsequent Fischer-Tropsch reaction. The reverse-water-gas-shift unit preferably comprises a catalyst comprising nickel. Such a catalyst is particularly suitable for carrying out a reverse-water-gas-shift reaction at favourable temperatures and pressures and at high conversion rates. The reverse-water-gas-shift unit is preferably at a temperature of at least 700° C. Such a temperature may result in a particularly high conversion rate of carbon dioxide and hydrogen to carbon monoxide and water.

The fixed-bed Fischer-Tropsch unit is provided downstream of a reverse-water-gas-shift unit. By “downstream” it is meant that the fixed-bed Fischer-Tropsch unit and reverse-water-gas-shift unit are in fluid communication and that during normal operation of the Fischer-Tropsch plant, gas flows to the reverse-water-gas-shift unit before flowing to the fixed-bed Fischer-Tropsch unit.

The method comprises providing a feed comprising hydrogen and carbon dioxide. The feed may comprise species other than hydrogen and carbon dioxide, for example water, carbon monoxide, methane and ammonia, as well as solid species such as, for example, dust and coke. The components of the feed gas may vary depending on its method of manufacture and the starting materials used. The feed may be generated by gasification of a carbonaceous feedstock, for example municipal solid waste. The hydrogen may be generated by electrolysis of water. The energy for the electrolysis may be from renewable sources, thereby rendering the method more environmentally friendly. The carbon dioxide may be a by-product of combustion. The carbon dioxide may be derived from flue gas. The carbon dioxide may derive from partial oxidation and/or from a gasifier. The feed may be subjected to one or more purification steps prior to being passed to the reverse-water-gas-shift unit, for example steps to remove one or more of water, carbon monoxide, methane, ammonia, and solid species. Suitable purification methods are known in the art.

The method comprises passing the feed to the reverse-water-gas-shift unit to produce a syngas enriched in carbon monoxide. As noted above, not all of the hydrogen is converted. This is because of the need for the syngas to contain hydrogen so as to undergo, for example, a subsequent Fischer-Tropsch reaction.

The term “syngas” or “synthesis gas” as used herein may encompass a fuel gas mixture. In the method of the present invention, the syngas comprises carbon monoxide (i.e., CO) and hydrogen (i.e., molecular hydrogen H₂). The syngas may comprise other species, for example water and/or unreacted carbon dioxide. By “enriched in carbon monoxide” it is meant that the syngas contains a higher amount of carbon monoxide than the feed. In other words, “passing the feed to the reverse-water-gas-shift unit to produce a syngas enriched in carbon monoxide” may encompass passing the feed to the reverse-water-gas-shift unit to convert at least some of the carbon dioxide and hydrogen to carbon monoxide and water.

The method comprises providing a first water source. The first water source may derive from the Fischer-Tropsch plant, such as process condensate and co-produced water. In such case, providing the water source may comprise subjecting the water source to a one or more purification steps. Alternatively, the first water source may be an external water source, such as boiler feed water.

The method comprises generating a first steam from the first water source using heat from the syngas to thereby form a cooled syngas. Heat from the syngas may be transferred to the first water source using, for example, a heat exchanger. During the generation of the first steam, the syngas and first water source are typically not in direct contact with each other. The cooled syngas is typically at a temperature suitable for it to be fed to a Fischer-Tropsch unit.

The method comprises passing at least some of the first steam to the Fischer-Tropsch unit to increase the temperature of the Fischer-Tropsch unit to a first operating temperature of the Fischer-Tropsch unit. The first operating temperature may be a temperature sufficient to undergo a Fischer-Tropsch reaction. The Fischer-Tropsch unit may be increased to the first operating temperature from ambient temperature.

Generating steam from the first water source using heat from the syngas preferably comprises passing the syngas to a steam-raising boiler coupled to a first steam drum. This may be a particularly efficient way of generating steam. Steam-raising boilers and steam drums are known in the art.

The first operating temperature of the Fischer-Tropsch unit is preferably from 100° C. to 130° C. At such a temperature cooled syngas passed to the Fischer-Tropsch unit is not expected to undergo a Fischer-Tropsch reaction, in which case the Fischer-Tropsch unit can be further heated to a higher second operating temperature (as described later).

At least some of the first steam that is passed to the Fischer-Tropsch unit is preferably passed to the Fischer-Tropsch unit at: a temperature of from 200 to 310° C., preferably from 240 to 290° C.; and/or a pressure of from 16 to 99 bara, preferably from 33 to 74 bara. Steam having such temperatures and/or pressures may be known in the art as “high-grade” steam. Such temperatures and pressures may be particularly suitable for heating the Fischer-Tropsch unit to the first operating temperature. Lower temperatures may result in the Fischer-Tropsch unit reaching the first operating temperature more slowly or not at all. Higher temperatures may result in the Fischer-Tropsch unit reaching an unfavourably high temperature. Lower pressures may be less efficient at heating the Fischer-Tropsch unit to the first operating temperature. Higher pressures may require more complicated or expensive equipment.

The reverse-water-gas-shift unit preferably comprises an autothermal reverse-water-gas shift unit (where heat for the endothermic reverse water-gas shift reaction is provided by combustion of a fuel with oxygen), an electrically-heated reverse-water-gas-shift unit, a plasma-heated reverse-water-gas-shift unit and/or a solid-oxide-electrolyser cell (SOEC). Such reverse-water-gas-shift units are known in the art and may be particularly suitable for converting the feed to syngas. Furthermore, such reverse-water-gas-shift units may be particularly suitable for generating heat to form the first steam.

The Fischer-Tropsch unit preferably comprises a catalyst comprising cobalt, iron and/or ruthenium, more preferably a cobalt-containing catalyst. Such a catalyst may be particularly effective at catalysing Fischer-Tropsch reactions and/or enable the reaction to proceed at favourably low temperatures and/or with high yield.

Preferably, the Fischer-Tropsch plant further comprises a carbon-dioxide-recovery unit downstream of the reverse-water-gas-shift unit and upstream of the Fischer-Tropsch unit; and the method further comprises passing at least some of the first steam to the carbon-dioxide-recovery unit to increase the temperature of the carbon-dioxide-recovery unit to a desired operating temperature of the carbon-dioxide-recovery unit. The carbon-dioxide-recovery unit may remove carbon dioxide from the cooled syngas before the cooled syngas is passed to the Fischer-Tropsch unit. This may result in an optimal syngas specification prior to feeding cooled syngas into the Fischer-Tropsch unit. Removing carbon dioxide from the cooled syngas may increase the efficiency of the method and a subsequent Fischer-Tropsch reaction using the cooled syngas. If not removed from the cooled syngas, carbon dioxide may accumulate to greater than 50 mol % and may adversely affect the performance of the Fischer-Tropsch catalyst. Heating the carbon-dioxide-recovery unit using steam from the reverse-water-gas-shift unit may enable the carbon-dioxide-recovery unit to reach an operating temperature without the use of an external source of steam. This reduces the complexity of the method. Since the first steam is generated using heat from the syngas, the energy requirement of the method is reduced, thereby reducing the cost and environmental impact. Carbon-dioxide-recovery units are known in the art. The carbon-dioxide-recovery unit preferably uses: a liquid chemical absorbent, more preferably selected from one or more of an amine (e.g. an alkyl amine selected from MEA, DEA and MDEA) or an alkali metal carbonate; and/or a liquid physical absorbent, more preferably selected from one or more of methanol, glycols or glycol ethers. Such absorbents may be particularly suitable for removing carbon dioxide. The liquid chemical absorbent may be part of a scrubber.

Preferably, the carbon-dioxide-recovery unit comprises an amine carbon-dioxide-recovery unit comprising an absorber unit and a regenerator unit comprising a regenerator column; the desired operating temperature of the carbon-dioxide-recovery unit corresponds to a desired operating temperature of the regenerator column bottoms; and the desired operating temperature is from 100° C. to 180° C. Such amine carbon-dioxide-recovery units are known in the art.

At least some of the first steam that is passed to the carbon-dioxide-recovery unit is preferably passed to the carbon-dioxide-recovery unit at: a temperature of from 170 to 230° C., more preferably from 180 to 220° C., even more preferably from 190 to 210° C.; and/or a pressure of from 5 to 25 bara, more preferably from 10 to 20 bara, even more preferably from 12 to 16 bara. Steam having such temperatures and/or pressures may be known in the art as “low-grade” steam. Such temperatures and pressures may be particularly suitable for heating the carbon-dioxide-recovery unit to the desired operating temperature. Lower temperatures may result in the carbon-dioxide-recovery unit reaching the desired operating temperature more slowly or not at all. Higher temperatures may result in the carbon-dioxide-recovery unit reaching an unfavourably high temperature. Lower pressures may be less efficient at heating the carbon-dioxide-recovery unit to the first operating temperature. Higher pressures may require more complicated equipment and/or reduce the safety of the method.

Preferably, at least some of the first steam that is passed to the carbon-dioxide-recovery unit is let down in pressure (for example, using a let-down valve) before being passed to the carbon-dioxide-recovery unit. This is because the steam pressure requirements of the carbon-dioxide-recovery unit may be lower than that of the Fischer-Tropsch unit. In other words, it may be preferable to supply “low-grade” steam to the carbon-dioxide-recovery unit. The supply of “high-grade” steam to the carbon-dioxide-recovery unit may result in the design temperatures and pressures of the carbon-dioxide-recovery unit being exceeded.

Preferably, from 30 to 50 mol. % of the first steam is passed to the Fischer-Tropsch unit and from 50 to 70 mol. % of the first steam is passed to the carbon-dioxide-recovery unit. This may ensure that the Fischer-Tropsch unit and carbon-dioxide-recovery unit reach their operating temperatures at suitable times during start-up.

The feed is preferably passed to the reverse-water-gas-shift unit at a temperature of from 400° C. to 1000° C., preferably from 450° C. to 900° C. Lower temperatures may mean that the reverse-water-gas-shift reaction occurs at an unfavourably low rate and/or low selectivity or not at all. Higher temperatures may increase the cost of the method without a corresponding increase in reaction rate and/or selectivity. To achieve such a temperature, the feed may be heated, for example, in an electric feed heater.

While the temperature of the Fischer-Tropsch unit is increased to the first operating temperature, preferably substantially none of the cooled syngas is passed to the Fischer-Tropsch unit. At temperatures below the operating temperature, it may be that the Fischer-Tropsch reaction does not occur or occurs only at a slow rate and/or with low selectivity.

In a further aspect, the present disclosure provides a method of starting up a Fischer-Tropsch plant and then operating the Fischer-Tropsch plant to produce a hydrocarbon product, the method comprising:
starting up a Fischer-Tropsch plant using the method of any preceding claim; and
once the Fischer-Tropsch plant has reached the first operating temperature, operating the Fischer-Tropsch plant by passing the cooled syngas to the Fischer-Tropsch unit to produce a hydrocarbon product.

The advantages and preferable features of the first aspect apply equally to this aspect.

The hydrocarbon product is preferably a liquid hydrocarbon product. The term “liquid hydrocarbon” as used herein may encompass species formed of carbon and hydrogen, including those that are liquid at room temperature and pressure. The hydrocarbons typically comprise alkanes, and typically comprise from 1 to 100 or higher carbon atoms per molecule. The liquid hydrocarbons preferably comprise alkanes. Alkanes may be a particularly desirable product. The hydrocarbon product may be subjected to one or more purification steps and/or refining steps and/or upgrading steps. Suitable purification steps, refining steps and upgrading steps are known in the art.

Passing the cooled syngas to the Fischer-Tropsch unit to produce a hydrocarbon product typically comprises contacting the cooled syngas with a Fischer-Tropsch catalyst contained within the Fischer-Tropsch unit.

The molar ratio of hydrogen to carbon monoxide in the cooled syngas is preferably from 1.8 to 2.2 since this is close to the stoichiometric ratio of the Fischer-Tropsch reaction of about 2.

During the step of operating the Fischer-Tropsch plant, the step of passing at least some of the first steam to the Fischer-Tropsch unit is preferably continued while the temperature of the Fischer-Tropsch unit is increased to a second operating temperature higher than the first operating temperature, wherein the second operating temperature is: at least 10° C. higher than the first operating temperature, preferably at least 15° C. higher than the first operating temperature, more preferably at least 20° C. higher than the first operating temperature; and/or from 135 to 250° C., preferably from 150 to 240° C. Between the first and second operating temperatures, the Fischer-Tropsch unit may be warm enough to undergo a Fischer-Tropsch reaction. However, such Fischer-Tropsch reaction may not be optimised with regard to rate, yield and/or selectivity. While the exothermic nature of the Fischer-Tropsch reaction may increase the temperature of the Fischer-Tropsch unit, continuing to heat the Fischer-Tropsch unit up to the second operating temperature may result in the second operating temperature being reached more quickly.

Preferably, once the Fischer-Tropsch unit reaches the second operating temperature, the step of passing at least some of the first steam to the Fischer-Tropsch unit is halted. At this point, the exothermic Fischer-Tropsch reaction may be occurring sufficiently to heat the Fischer-Tropsch unit to a final desired operating temperature. At this point, the first steam may then be passed to other “high-grade” steam users in the Fischer-Tropsch plant. The second operating temperature may be sufficient to generate steam in a steam boiler coupled to the Fischer-Tropsch unit. The final desired operating temperature is preferably less than or equal to 300° C. Higher temperatures may increase the energy cost of the method without a significant increase in the levels of liquid hydrocarbons being produced.

Preferably, the volume of the first steam passed to the Fischer-Tropsch unit is gradually reduced between the first and second operating temperatures. The term “gradually reduced” as used herein may encompass multiple reduction steps, e.g., at least two reduction steps, or may encompass a continuous reduction as the temperature increases. As the temperature increases from the first to the second operating temperature, the exothermic Fischer-Tropsch reaction will ramp up, meaning that the need to heat using the first steam is gradually reduced.

Preferably, the Fischer-Tropsch plant further comprises a carbon-dioxide-recovery unit downstream of the reverse-water-gas-shift unit and upstream of the Fischer-Tropsch unit; starting up the Fischer-Tropsch plant comprises passing at least some of the first steam to the carbon-dioxide-recovery unit to increase the temperature of the carbon-dioxide-recovery unit to a desired operating temperature of the carbon-dioxide-recovery unit; and operating the Fischer-Tropsch plant further comprises passing the cooled syngas to the carbon-dioxide-recovery unit to recover carbon dioxide from the cooled syngas prior to passing the cooled syngas to the Fischer-Tropsch unit. The advantages and preferable features of the carbon-dioxide-recovery unit are as described above.

The method preferably further comprises recycling carbon dioxide recovered from the carbon-dioxide-recovery unit to the feed. This may avoid releasing carbon dioxide to the atmosphere and may increase the “carbon efficiency” of the method.

Operating the Fischer-Tropsch plant preferably further comprises: providing a second water source; generating a second steam from the second water source using heat from the Fischer-Tropsch unit; and passing at least some of the second steam to the carbon-dioxide-recovery unit to maintain the operating temperature of the carbon-dioxide-recovery unit. This may avoid the need for an external source of steam and the corresponding disadvantages described above. Such second steam is typically “low-grade” and is therefore suitable for passing to the carbon-dioxide-recovery unit.

Preferably, the Fischer-Tropsch unit comprises a tube side and a shell side; the second water source is provided on the shell side; and generating the second steam from the second water source using heat from the Fischer-Tropsch unit comprises the use of a second steam drum coupled to the shell side. By “tube side” it is meant the interior of tubes inside a vessel in which the Fischer-Tropsch reaction is taking place. By “shell side” it is meant the exterior side of those same tubes within the vessel i.e. the space inside the vessel between the tubes and between the tubes and the inside wall of the vessel. By providing the second water source on the shell side and generating steam therefrom, the Fischer-Tropsch unit may be cooled. Since the Fischer-Tropsch reaction is exothermic, this may ensure that the Fischer-Tropsch unit does not reach an unfavourably high operating temperature.

Preferably, generating steam from the first water source using heat from the syngas comprises passing the syngas to a steam-raising boiler coupled to a first steam drum; and the first steam drum generates steam at a higher pressure than the second steam drum. Typically, the first steam drum generates “high-grade” steam (since the reverse-water-gas-shift unit typically operates at a relatively high temperature), whereas the second steam drum generates “low-grade” steam (since the Fischer-Tropsch unit typically operates at a relatively low temperature).

The cooled syngas passed to the Fischer-Tropsch unit preferably comprises less than 50 mol. % carbon dioxide, more preferably less than 30 mol. % carbon dioxide, even more preferably less than 20 mol. % carbon dioxide, still even more preferably less than 10 mol. % carbon dioxide, still even more preferably less than 5 mol. % carbon dioxide, still even more preferably less than 1 mol. % carbon dioxide, based on the total moles of the cooled syngas passed to the Fischer-Tropsch unit. As discussed above, higher levels of carbon dioxide may adversely affect the performance of the Fischer-Tropsch catalyst and may decrease the efficiency of the method.

Once the Fischer-Tropsch unit reaches the second operating temperature, the step of passing at least some of the first steam to the carbon-dioxide-recovery unit is preferably halted. At this point, the first steam may then be passed to other “high-grade” steam users in the Fischer-Tropsch plant. The second operating temperature may be sufficient to generate “low-grade” steam in a steam boiler coupled to the Fischer-Tropsch unit, which may be passed to the carbon-dioxide-recovery unit top maintain the temperature of the carbon-dioxide-recovery unit during operation of the Fischer-Tropsch plant.

Preferably, once the Fischer-Tropsch unit reaches the second operating temperature, at least some of the first steam is passed to one or both of: a Fischer-Tropsch unit preheater and a purification bed preheater. This may avoid the need for an external source of steam to heat such preheaters and the corresponding disadvantages.

The present invention may be used to restart a process that has been temporarily shut down or may be used at initial start-up of the Fischer-Tropsch unit where the Fischer-Tropsch catalyst requires activation.

The invention will now be described in relation to the following non-limiting example.

### Example

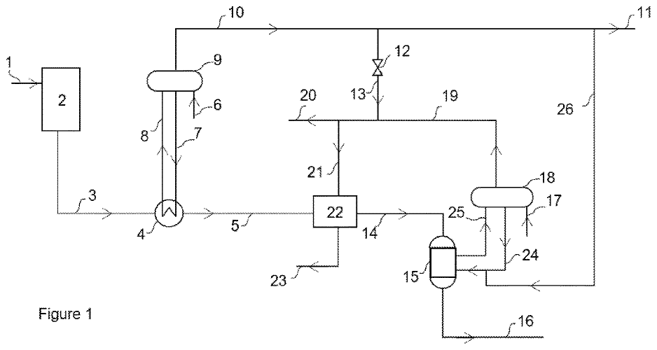

An example of a method according to the present invention is described with reference to FIG. 1. It will be understood by those skilled in the art that the drawings are diagrammatic and that further items of equipment such as reflux drums, compressors, pumps, vacuum pumps, towers, heat exchangers, temperature sensors, pressure sensors, pressure relief valves, control valves, flow controllers, level controllers, holding tanks, storage tanks, and the like may be required in a commercial plant. The provision of such ancillary items of equipment forms no part of the present invention and is in accordance with conventional chemical engineering practice.

In FIG. 1, a feed comprising of hydrogen and carbon dioxide via line 1 is passed to the inlet of a reverse-water-gas-shift unit 2 to produce syngas enriched in carbon monoxide 3. The hot syngas then passes through a syngas boiler 4, to generate high-grade steam 10 via the close-coupled HP Steam Drum 9. The HP Steam Drum 9 is fed by boiler feed water 6, which circulates to the syngas boiler 4 via the downcomers 7 and risers 8 by natural circulation. During normal operation, the high-grade steam 10 is sent to the high-grade steam users via line 11.

The cooled syngas 5 is sent to the carbon-dioxide-recovery unit 22 to remove carbon dioxide to achieve the correct syngas specification to the Fischer-Tropsch unit 15 The carbon dioxide 23 is recycled to the reverse-water-gas-shift unit 2. The syngas stream 14, enters the tube side of the Fischer-Tropsch unit 15. The Fischer-Tropsch synthesis reaction is an exothermic reaction. The heat generated from the Fischer-Tropsch unit is used to raise low-grade steam in the shell side of the Fischer-Tropsch unit 15 via a LP Steam Drum 18. The LP Steam Drum 18 is supplied by boiler feed water 17 and coupled to the Fischer-Tropsch unit 15 via downcomers 24 and risers 25. During normal operation, the low-grade steam 19 is sent to the low-grade steam users via line 20 and the carbon-dioxide-recovery unit 22 via line 21. The low-grade steam is used to provide the heat for the re-boiling duty of the amine regeneration unit (not shown) in the carbon-dioxide-recovery unit 22.

At start-up, the temperature of the Fischer-Tropsch unit 15 is increased by heating with high-grade steam from the HP Steam Drum 9 via line 26. High-grade steam is added to the shell side of the Fischer-Tropsch unit 15 to raise the temperature in the Fischer-Tropsch unit 15 to initiate Fischer-Tropsch reaction. Without the Fischer-Tropsch synthesis reaction, there is no low-grade steam generation in the LP Steam Drum 18 to provide the low-grade steam users and carbon-dioxide-recovery unit 22. At start-up, the heat to the low-grade steam users is supplied by letting down the high-grade steam via a let-down valve 12 to the low-grade steam users' pressure. This enables the start-up of the carbon-dioxide-recovery unit 22 to achieve the correct syngas specification to the Fischer-Tropsch unit 15. When the Fischer-Tropsch unit 15 has reached operating temperature and raising sufficient low-grade steam to the low-grade steam users, the high-grade steam let-down 12 valve is closed.

The foregoing detailed description has been provided by way of explanation and illustration and is not intended to limit the scope of the appended claims. Many variations in the presently preferred embodiments illustrated herein will be apparent to one of ordinary skill in the art and remain within the scope of the appended claims and their equivalents.

1-24. (canceled)

25. A method of starting-up a Fischer-Tropsch plant, the method comprising:
providing a Fischer-Tropsch plant comprising a fixed-bed Fischer-Tropsch unit downstream of a reverse-water-gas-shift unit;
providing a feed comprising hydrogen and carbon dioxide;
passing the feed to the reverse-water-gas-shift unit to produce a syngas enriched in carbon monoxide;
providing a first water source;
generating a first steam from the first water source using heat from the syngas to thereby form a cooled syngas;
passing at least some of the first steam to the Fischer-Tropsch unit to increase the temperature of the Fischer-Tropsch unit to a first operating temperature of the Fischer-Tropsch unit.

26. The method of claim 25, wherein generating steam from the first water source using heat from the syngas comprises passing the syngas to a steam-raising boiler coupled to a first steam drum.

27. The method of claim 25, wherein the at least some of the first steam that is passed to the Fischer-Tropsch unit is passed to the Fischer-Tropsch unit at:
a temperature of from 200 to 310° C., preferably from 240 to 290° C.; and/or
a pressure of from 16 to 99 bara, preferably from 33 to 74 bara.

28. The method of claim 25, wherein:
the Fischer-Tropsch plant further comprises a carbon-dioxide-recovery unit downstream of the reverse-water-gas-shift unit and upstream of the Fischer-Tropsch unit; and
the method further comprises passing at least some of the first steam to the carbon-dioxide-recovery unit to increase the temperature of the carbon-dioxide-recovery unit to a desired operating temperature of the carbon-dioxide-recovery unit.

29. The method of claim 28, wherein:
the carbon-dioxide-recovery unit comprises an amine carbon-dioxide-recovery unit comprising an absorber unit and a regenerator unit comprising a regenerator column;
the desired operating temperature of the carbon-dioxide-recovery unit corresponds to a desired operating temperature of the regenerator column bottoms; and
the desired operating temperature is from 100° C. to 180° C.

30. The method of claim 28, wherein the at least some of the first steam that is passed to the carbon-dioxide-recovery unit is let down in pressure before being passed to the carbon-dioxide-recovery unit.

31. The method of claim 28, wherein from 30 to 50 mol. % of the first steam is passed to the Fischer-Tropsch unit and from 50 to 70 mol. % of the first steam is passed to the carbon-dioxide-recovery unit.

32. The method of claim 25, wherein while the temperature of the Fischer-Tropsch unit is increased to the first operating temperature, substantially none of the cooled syngas is passed to the Fischer-Tropsch unit.

33. A method of starting up a Fischer-Tropsch plant and then operating the Fischer-Tropsch plant to produce a hydrocarbon product, the method comprising:
starting up a Fischer-Tropsch plant using the method of any preceding claim; and
once the Fischer-Tropsch plant has reached the first operating temperature, operating the Fischer-Tropsch plant by passing the cooled syngas to the Fischer-Tropsch unit to produce a hydrocarbon product.

34. The method of claim 33, wherein during the step of operating the Fischer-Tropsch plant, the step of passing at least some of the first steam to the Fischer-Tropsch unit is continued while the temperature of the Fischer-Tropsch unit is increased to a second operating temperature higher than the first operating temperature, wherein the second operating temperature is:
at least 10° C. higher than the first operating temperature, preferably at least 15° C. higher than the first operating temperature, more preferably at least 20° C. higher than the first operating temperature; and/or
from 135 to 250° C., preferably from 150 to 240° C.

35. The method of claim 33, wherein once the Fischer-Tropsch unit reaches the second operating temperature, the step of passing at least some of the first steam to the Fischer-Tropsch unit is halted.

36. The method of claim 34, wherein the volume of the first steam passed to the Fischer-Tropsch unit is gradually reduced between the first and second operating temperatures.

37. The method of claim 33, wherein:
the Fischer-Tropsch plant further comprises a carbon-dioxide-recovery unit downstream of the reverse-water-gas-shift unit and upstream of the Fischer-Tropsch unit;
starting up the Fischer-Tropsch plant comprises passing at least some of the first steam to the carbon-dioxide-recovery unit to increase the temperature of the carbon-dioxide-recovery unit to a desired operating temperature of the carbon-dioxide-recovery unit; and
operating the Fischer-Tropsch plant further comprises passing the cooled syngas to the carbon-dioxide-recovery unit to recover carbon dioxide from the cooled syngas prior to passing the cooled syngas to the Fischer-Tropsch unit.

38. The method of claim 37, further comprising recycling carbon dioxide recovered from the carbon-dioxide-recovery unit to the feed.

39. The method of claim 33, wherein operating the Fischer-Tropsch plant further comprises:
providing a second water source;
generating a second steam from the second water source using heat from the Fischer-Tropsch unit; and
passing at least some of the second steam to the carbon-dioxide-recovery unit to maintain the operating temperature of the carbon-dioxide-recovery unit.

40. The method of claim 39, wherein:
the Fischer-Tropsch unit comprises a tube side and a shell side;
the second water source is provided on the shell side;
generating the second steam from the second water source using heat from the Fischer-Tropsch unit comprises the use of a second steam drum coupled to the shell side.

41. The method of claim 40, wherein:
generating steam from the first water source using heat from the syngas comprises passing the syngas to a steam-raising boiler coupled to a first steam drum; and
the first steam drum generates steam at a higher pressure than the second steam drum.

42. The method of claim 37, wherein the cooled syngas passed to the Fischer-Tropsch unit comprises less than 50 mol. % carbon dioxide, preferably less than 30 mol. % carbon dioxide, more preferably less than 20 mol. % carbon dioxide, even more preferably less than 10 mol. % carbon dioxide, still even more preferably less than 5 mol. % carbon dioxide, still even more preferably less than 1 mol. % carbon dioxide, based on the total moles of the cooled syngas passed to the Fischer-Tropsch unit.

43. The method of claim 37, wherein once the Fischer-Tropsch unit reaches the second operating temperature, the step of passing at least some of the first steam to the carbon-dioxide-recovery unit is halted.

44. The method of claim 33, wherein once the Fischer-Tropsch unit reaches the second operating temperature, at least some of the first steam is passed to one or both of: a Fischer-Tropsch unit preheater and a purification bed preheater.
